(19) United States
(12) Patent Application Publication
(10) Pub. No.: US 2015/0080769 A1
(43) Pub. Date: Mar. 19, 2015

(54) DEVICE FOR CRYOLIPOLYSIS

(71) Applicant: Friedemann LOTSCH, (US)

(72) Inventor: Friedemann Lotsch, Dortmund (DE)

(21) Appl. No.: 14/383,236

(22) PCT Filed: Mar. 7, 2013
(86) PCT No.: PCT/EP2013/000678

§ 371 Date: Sep. 15, 2014

(30) Foreign Application Priority Data
Mar. 8, 2012 (DE) 20 2012 002 278.4

(51) Int. Cl. A61F 7/10; A61N 7/00

(52) U.S. Cl. CPC A61F 7/10; A61N 7/00; A61F 2007/0056
USPC 601/2; 607/96; 607/104

(57) ABSTRACT

The invention relates to an applicator for use with a device for treating subcutaneous lipid-rich cells by cold treatment, wherein the applicator (1) comprises a contact surface (2) that is formed as a surface (3) of a solid treatment plate (4) made of aluminum or another material having good heat conductivity. The invention further relates to a device for treating subcutaneous, lipid-rich cells by cold treatment comprising a heat transfer circuit and an applicator (1) through which a heat transfer medium (6) flows for treating a live body. The device consists of a refrigeration device (30) for providing the heat transfer medium (6) having a specific temperature and connecting supply lines (29) to the applicator as well as a compensator (31) that compensates the weight of the applicator.

The invention relates to an applicator for use in connection with a device for the treatment of subcutaneous, fat-rich cells using cold.

Furthermore, the invention relates to a device for the treatment of subcutaneous, fat-rich cells using cold.

It is known that for physical/medical treatment of specific symptoms, for example in rheumatology and sports medicine, the body parts in question are cooled, whereby different methods are used, according to the state of the art.

Thus, it is usual to spray gases from pressurized containers onto the skin for medical cold treatment, and to thereby achieve rapid and effective cooling results. In this connection, the work is performed with physiologically problematic gases, such as propane, which are not allowed to be inhaled, are dermatologically problematic, and furthermore represent a fire hazard.

Furthermore, an apparatus for partial icing of surfaces, particularly for the treatment of superficial skin symptoms, is known, whereby the handling device is equipped with Peltier modules. In this connection, the treatment body that is passed out of the handling device is connected, in heat-conductive manner, with the cold surface of a Peltier module. The Peltier module itself is mounted on a heat distribution plate and connected to a heat exchanger, the cooling circuit of which is connected with the energy provision unit and/or a cooling water connector, by means of a piping circuit. As a result of the given combination with embedding of a Peltier element, the handling device in question is correspondingly heavy and also complicated in terms of its structure. Furthermore, increased energy expenditure is required as a result of the energy emission or transfer over multiple exchange surfaces.

An improvement has been achieved, in a known apparatus, in which the treatment probe itself is configured as a relaxation and/or flow-through space for the refrigerant. In this connection, the treatment probe has been structured as a tubular body with a relaxation and/or flow-through space and a front treatment body. By means of the direct supply of coolant into the flow-through space or the relaxation of the refrigerant in the relaxation space of the treatment probe, a significantly better cooling effect is created, and thereby a much lower supply of energy is required. Furthermore, a particularly handy and light device is created, by means of the elimination of the Peltier elements that are otherwise usual.

Furthermore, very precise temperature determination and an immediate reaction of the treatment probe are ensured.

Furthermore, a treatment probe is known, which consists of an energy part that is connected with a treatment head and/or can be connected interchangeably, and the treatment head consists of an elastic surface.

The known applicators are generally adapted very specifically to the medical application case, in each instance. The therapeutic goal determines the temperature level, the treatment period, and the treatment location. In order to meet these requirements, flexible applicators are known from DE 296 11 440 U1, for example. Cuffs made from plastic, which adapt to the shape of the body, are also commercially available.

A disadvantage of flexible cuffs is the low heat transfer that results from the use of plastics. Rigid applicators, on the other hand, do not lie closely against the shape of the body, so that their use is often prohibited if only for this reason. However, if they are specially weakened mechanically so that they can lie closely against the surface of a body to a certain extent, as proposed in DE 296 11 440 U1, this leads to significant production effort.

For treatment of subcutaneous, fat-rich cells as described in WO 2010/127315, for example, combined designs are used, because precise temperature guidance is important. The special requirements in this area are the object of WO 2007 133839 A1, WO 2008 039556 A1, WO 2009 011708 A1.

It is the task of the invention to ensure significantly improved surface contact also for large-area sections and parts of the body, while maintaining an optimal cooling effect, and, in this connection, to allow the most advantageous heat dissipation possible.

[A01] In the case of an applicator for use in combination with a device for the treatment of subcutaneous, fat-rich cells using cold, this task is accomplished in that the applicator has a contact surface that is shaped as the surface of a rigid treatment plate, preferably from aluminum. By means of the rigid plate, the conditions of use become advantageously reproducible, and the process can be more precisely controlled. At the same time, the body heat is more rapidly extracted from the body by means of the selection of a material that has particularly good heat conductivity.

[A02] In order for the treatment plate to maintain a uniform, low temperature, it is provided that channels for conducting a heat medium are provided in the rigid treatment plate. Preferably, a brine having a supply temperature that is precisely regulated and amounts to approx. 2° Celsius is used as the heat medium. The controller determines the amount of heat conducted away from the temperature difference between the supply and the return, and from the mass stream. The channels can be provided in the interior of the plate or as covered ditches in the surface of the plate.

[A03] Because the rigid treatment plate is connected with a further rigid plate, layered to form a stack, the mechanical strength is increased and a more uniform temperature distribution occurs.

[A04] The channels for the heat medium for cooling the treatment plate can be produced in particularly advantageous manner if the channels run in a boundary surface of the two firmly connected plates. The channels are then cut into one or both plates as open grooves, at first. After the plates have been joined, closed channels are then formed. These can be distributed over the entire plate in meander shape, in order to achieve uniform cooling of the entire surface.

Pipes or hoses can also be introduced into the channels.

[A05] The measure that an outer insulation layer that covers the stack is provided also serves for improved cooling. The layer can advantageously consist of a compacted polyurethane foam that provides a shield against parasitic heat streams into the treatment plate from the surroundings, so that the determination of the amounts of heat conducted away from the body also becomes more precise. This can be further improved if a gap is provided between the insulation layer and the further rigid plate. If the gap is furthermore configured to have a vacuum applied to it, heat transfer is reduced even further by means of the lower gas density in the gap. In addition, it is advantageous if a connection is provided between contact surface and gap, so that the body surface to be treated is also drawn against the contact surface as the result of the vacuum. In this way, a partial vacuum occurs in the space between the contact surface and the surface to be treated, which leads to a secure hold of the applicator on the body part. It is advantageous if the outer insulation layer is formed into a housing by means of corresponding shaping, so that it also takes on the function of a device housing.

[A06] For secure placement of the treatment plate onto a body part, the applicator has at least one opening disposed in or around the contact surface, to which opening a partial vacuum is applied. As a result, the plate is drawn against the skin of the body, as soon as a partial vacuum is applied to the opening. This leads to particularly well reproducible contact of the surface with the skin.

[A07] The measure that the opening to which a partial vacuum is applied is disposed to end in a circumferential groove that frames the contact surface also serves to improve contact of the treatment plate. The circumferential groove draws the air under the contact surface away more rapidly.

[A03] It is advantageous if the groove is disposed in a rigid frame that encloses the layers. Then, only a connection line to the frame, to which a partial vacuum is applied, is required, along with supply and return lines to the treatment plate for the heat medium.

[A09] For secure contact of the treatment plate, it is furthermore provided that a circumferential gasket that frames the contact surface is provided, which gasket can preferably be easily replaced. Leakage flows are advantageously reduced, so that the partial vacuum unit can be designed to have lower power, and unnecessary noises are avoided. The gasket delimits the space between contact surface and body surface to be treated, toward the outside, so that the space formed in this way can also have a partial vacuum applied to it with low suction power, because no major leakage streams occur. Because the gasket is preferably removable, it can be easily cleaned and disinfected, if necessary. Just like the contact surfaces that lie below the gasket.

[A10] Because the gasket is shaped to partially cover the enclosed contact surface in the direction of the surface, and to run out in wedge shape, and because channels directed transverse to the course of the gasket are provided in the transition region, the possibility exists of treating even body parts that have a greater curvature, using the same applicator. By means of the shaping, an overly great contact surface is reduced. In the case that connection channels to the partial vacuum region end in the periphery of the gasket, at least one channel directed transverse to the course of the gasket can be provided in the transition region, which channel guides the partial vacuum into the space between contact surface and body surface to be treated. The gasket thereby comes to lie closely against the body surface. The treatment surface makes contact with the contact surface as a result of the partial vacuum.

[A11] The treatment can be comprehensively documented, because a temperature sensor is provided, which is preferably disposed on the contact surface or built into the cooling plate. This sensor provides the signal to the controller for shutting off cooling if the temperature drops below a threshold value, for example, and/or for documenting the skin temperature directly.

[A12] In a further embodiment, an ultrasound vibrator is provided in the treatment plate. In this way, the tissue can be additionally stimulated, and the effect of the applicator can be advantageously reinforced even more. If the treatment plate is configured to be concave in at least one direction, contact with body surfaces having different curvatures, particularly on the abdomen, the hip, the thighs, etc., is further improved.

[A13] In the treatment of the insides of the thighs, it is advantageous if two treatment plates are connected with one another to form a stack with contact surfaces disposed on both sides. Then, both inner thigh surfaces can be treated at the same time, in that the applicator is wedged in between the thighs.

[A14] The applicator can advantageously also have a housing, the interior of which is configured to have a vacuum applied to it, and in which supply lines end in vacuum-tight manner. In this manner, the housing simultaneously takes on the function of a partial vacuum distributor. By means of the suitable placement of connection lines that lead into the interior of the housing, the partial vacuum can also be passed into the space adjacent to the contact surface. The weight of the applicator is not necessary for its secure hold on the surface to be treated. Instead, the partial vacuum produces the required holding force. For this reason, the weight of the applicator can be compensated by a balancer, so that it is easy to handle.

[A15] The task is also accomplished by a device for the treatment of subcutaneous, fat-rich cells using cold, which device consists of a heat medium circuit and an applicator through which a heat medium flows, according to at least one of the preceding claims, and with a refrigeration device for making the heat medium available at a specific temperature, and with connecting supply lines to the applicator, as well as a balancer that compensates the weight of the applicator. In this connection, any apparatus that compensates the weight of the applicator is a balancer.

A preferred embodiment of the invention will be explained as an example, using a drawing. The figures of the drawing show, in detail:

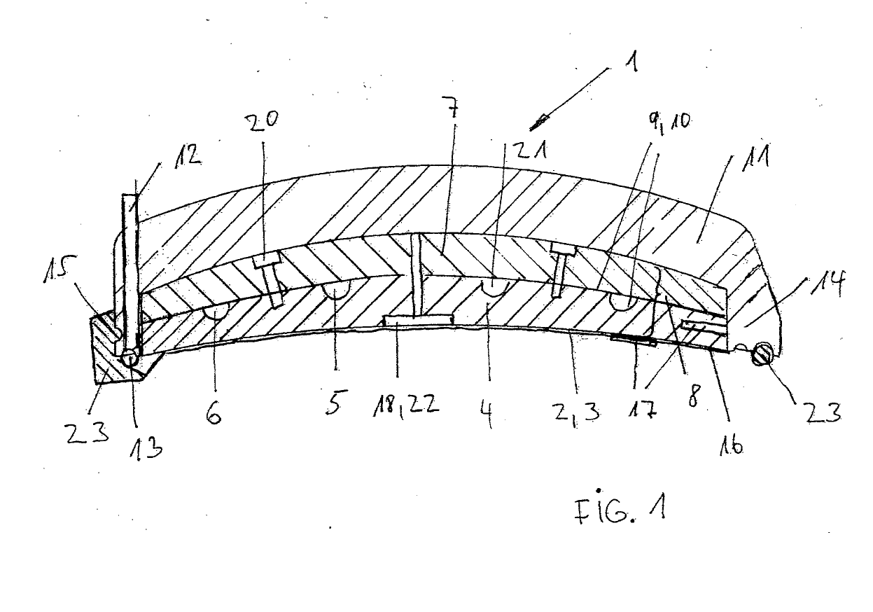

FIG. 1 a schematic cross-section through the applicator,

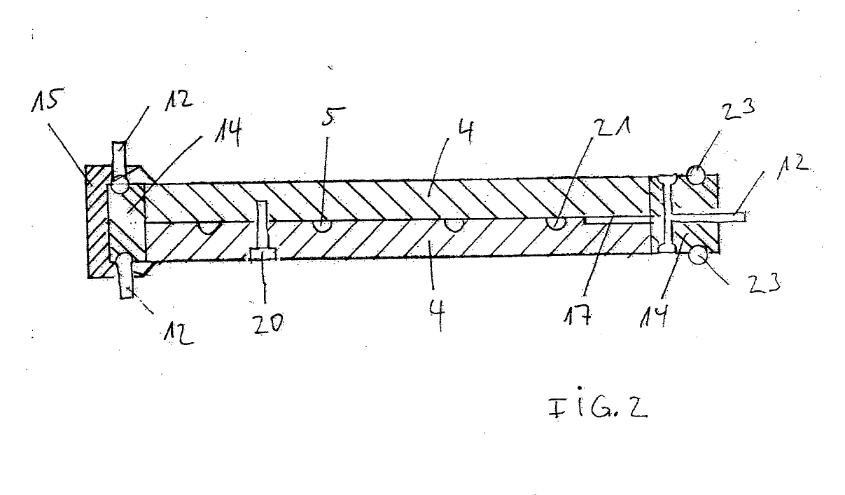

FIG. 2 a schematic cross-section through a further embodiment of the applicator according to the invention,

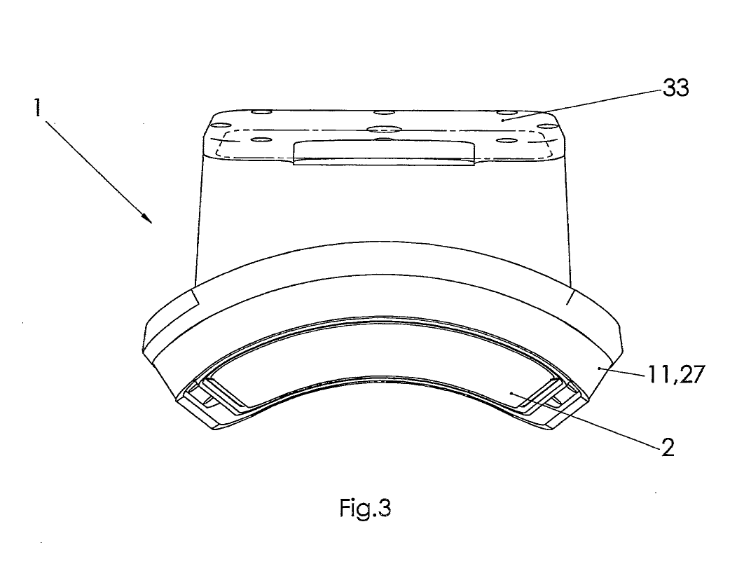

FIG. 3 a side view of a leg applicator,

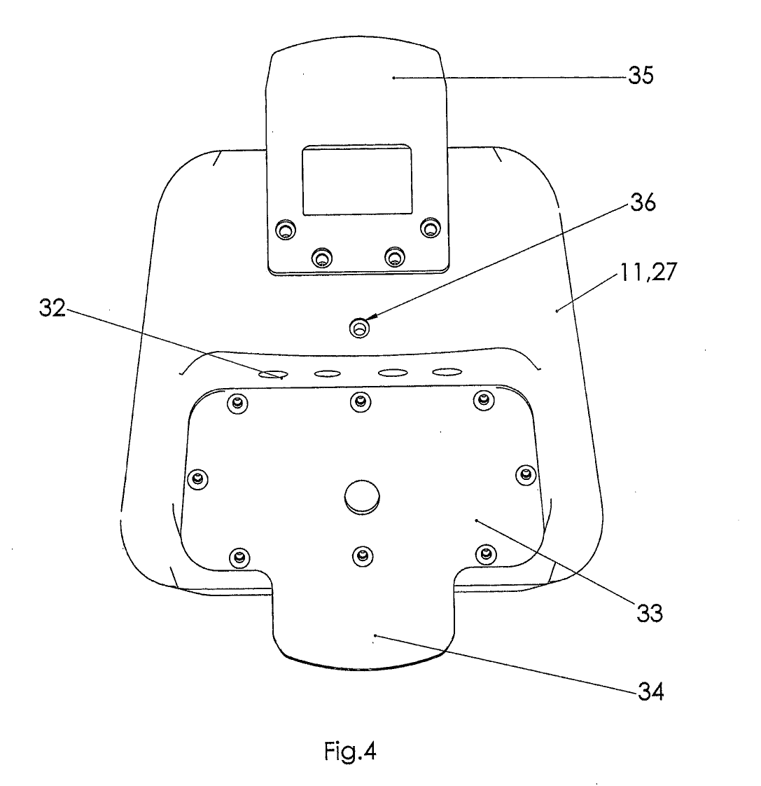

FIG. 4 a top view of a leg applicator according to FIG. 3,

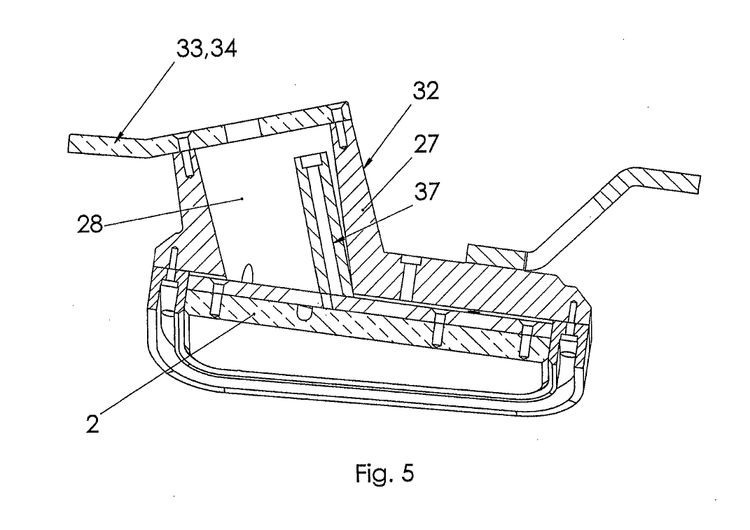

FIG. 5 a vertical section through a leg applicator according to section line A-A in FIG. 3,

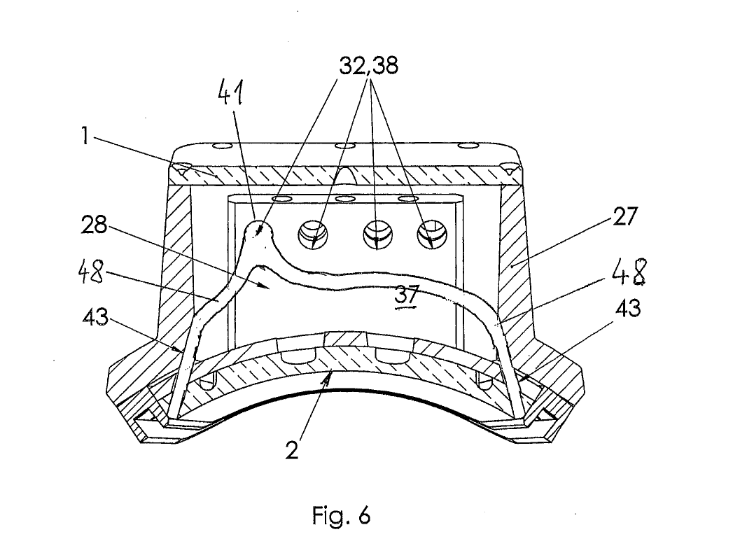

FIG. 6 a vertical section through a leg applicator according to section line VI-VI in FIG. 5,

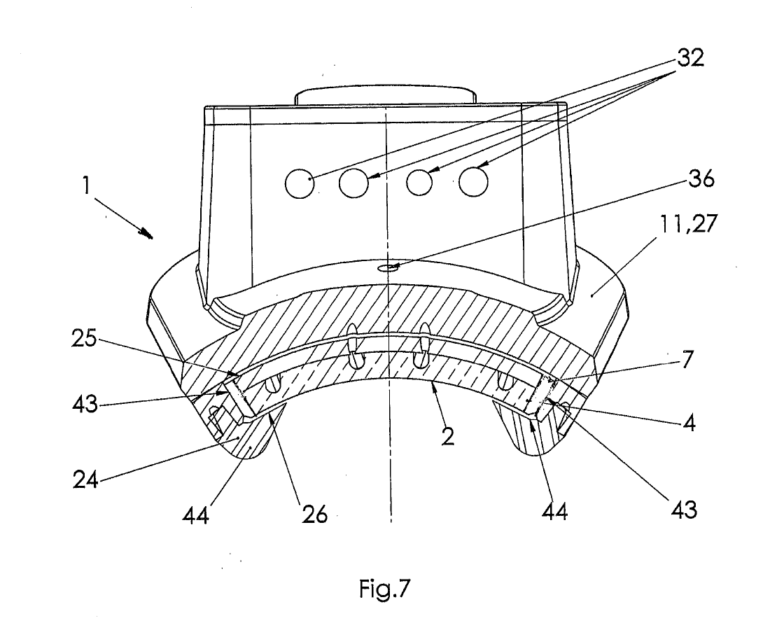

FIG. 7 a vertical section through a leg applicator according to section line VII-VII in FIG. 5, and

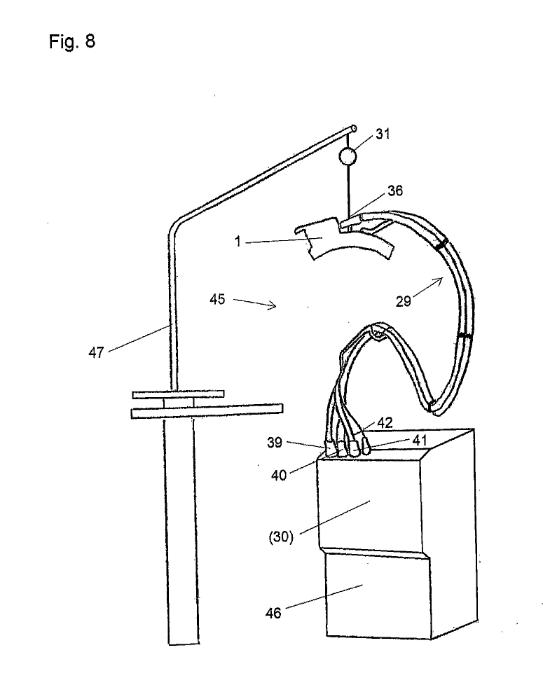

FIG. 8 a perspective view of the device.

In FIG. 1, the cross-section through a preferred embodiment of the applicator 1 according to the invention is shown. This applicator consists of a rigid treatment plate 4, the surface 3 of which, in use, represents the contact surface 2 with the skin of the person to be treated. In order for the body heat to be conducted away with the least possible heat conduction resistance, the preferred material of this treatment plate 4 is a good heat conductor, such as that represented by aluminum, for example. This treatment plate 4 is covered by a further rigid plate 7, which is connected with the plate 7 to form a stack 8, by means of multiple screws 20. The plate 7 can consist of the same material as the treatment plate 4, in order to prevent tensions caused by temperature differences and by different expansion coefficients. Preferably, however, the plate 7 can also be produced from a plastic, for example a plastic reinforced with glass fibers, in order to give the entire plate greater mechanical strength and to nevertheless reduce the transfer of heat to the outside. The body heat is conducted into a heat medium by way of the contact surface 2 and the treatment plate 4. The heat medium flows through the channels 5 in forced circulation. For this purpose, depressions are provided in one or both plates 7, 4, in suitable manner. After the two plates 4, 7 are joined together to form the stack 8, channels having a flow path that cools the treatment plate 4 uniformly are formed on the boundary surfaces 9, 10. In order not to have to dissipate any parasitic heat from the surrounding room air, by means of the heat medium 21, an insulation layer 11 that covers the stack is provided as a third, outer layer, which consists, for example, of a compacted hard polyurethane foam and is provided with a film or paint on the outside, in optically appealing manner.

To support the effect, one or more ultrasound vibrators 18 and/or stimulus current electrodes can also be additionally provided in the treatment plate. The three layers are enclosed, toward the outside, by a frame 14, which also serves as a mechanical support for the holder of the connection fittings for supplying the heat medium to the channels 5. In the left section of the frame, an opening 12 is additionally shown, which opens toward the contact surface 2 and has a partial vacuum applied to it. The opening 12 ends in a groove 13, which is cut into the lower circumferential surface of the frame. If the applicator described in this manner is laid onto the skin of a human body, and if sufficient partial vacuum is produced in the opening 12, the applicator itself is drawn against the body surface and produces an excellent heat transfer to the body tissue. In addition, the applicator can also be enclosed by a further circumferential gasket, in order to reduce or exclude a leakage air flow that might occur. On the right side of FIG. 1, an alternative embodiment of the frame, having a beaded gasket 24, is shown.

The adapter plate according to the invention cannot cool the body tissue below the temperature of the heat medium. It is consequently sufficient if the temperature of the heat medium is regulated precisely. Nevertheless, it can be advantageous if a temperature sensor 17, for example in the form of a film sensor, is disposed on the surface 3 of the treatment plate 4. The film sensor prevents heat transfer only insignificantly, but does measure the actual current tissue temperature at the surface 3. The signal of this sensor can be used for record-keeping and regulation of the course of treatment, for example.

A nonwoven fabric 16, which separates the surface 3 of the treatment plate from the body surface, particularly also for hygienic reasons, and is replaceable, serves to further increase the convenience during treatment. If the nonwoven fabric is air-permeable, it can be configured to also cover the gasket, to improve the hygiene.

FIG. 2 shows a special embodiment, in which two treatment plates have been joined together to form a stack. Such an applicator is used, for example, in the treatment of the inner surfaces of the thighs. This applicator can then be wedged between the legs. In this embodiment, the insulation layer 11 and the plate 7 are not necessary. For the remainder, the method of functioning of this applicator is analogous to the method of functioning described in connection with FIG. 1.

FIGS. 3 to 7 show a further exemplary embodiment of the applicator 1 according to the invention. There, the insulation layer 11 is shaped to form a housing 27, into which openings 32 are introduced for connection fittings for supplying the heat medium and for connecting any electrical sensors or ultrasound vibrators. Two of the openings 32 serve for installation of the connection fittings for inflow and return of the heat medium. One opening is available for the connection fitting of a vacuum hose, and a fourth opening serves for installation of electrical plug connectors. The interior of the housing 27 is accessible behind a lid 33 that can be screwed on and is simultaneously shaped as a handle 34. By means of a further handle 35, the applicator can easily be moved with two hands, despite its weight. In this connection, the contact surface 2 is bent in concave shape transverse to the axis of symmetry, in order to lay the applicator 1 onto a thigh, for example. A centrally disposed attachment point 36 serves for suspending the applicator 1 on a balancer 31 (FIG. 8).

FIG. 5 shows a vertical section along the axis of symmetry of the housing. An attachment plate 37 is disposed behind the openings 32, with corresponding installation openings 38. These installation openings 38 are disposed behind the openings 32 of the housing, and serve as attachment points for the heat transfer inflow hose 39, the return 40 of the heat medium, the vacuum hose 41, and the cable 42.

In FIG. 6, a variant of the applicator 28 is shown, in which the interior 28 of the housing 27 does not have a partial vacuum applied to it. Instead, the partial vacuum is distributed by the vacuum hose 41, among two connection hoses 48 that pass the partial vacuum on to the connection channels 43.

Alternatively, the interior 28 of the housing 27 can be reduced in pressure, as compared with the atmosphere, by means of the vacuum hose 41. Then the connection hoses 48 to the connection channels 43, which connect the interior 28 with the space enclosed by the circumferential gasket and adjacent to the contact surface 2 are eliminated. As a result, the applicator is drawn against the body surface that lies underneath.

In the vertical section transverse to the axis of symmetry according to FIG. 7, it can be seen that a gap 25 is provided between the treatment plate 4 with the additional plate 7 and the insulation layer 11, which gap stands in connection with the interior 28 of the housing 27, and in which a partial vacuum therefore also prevails. From there, the partial vacuum gets to the contact surface 2 by way of connection channels 43.

The beaded gasket shown in FIG. 7 runs out in wedge shape in a transition region 26, toward the contact surface 2. In this transition region 26, it therefore covers the contact surface 2 and makes it smaller. The adapter 1 can consequently be adapted to body surfaces having a greater curvature, as well. In this connection, channels 44 in the gasket ensure that the partial vacuum reaches the contact surface 2.

Finally, the device 45 is shown in FIG. 8. It consists of the refrigeration device 30 (covered) for making available the heat medium, disposed in the housing block 46, of the connecting supply lines 29 to the adapter 1, and of the balancer 31, which keeps the adapter 1 ready for use at the holder 47, in essentially weightless manner.

In this manner, an applicator 1 for the treatment of subcutaneous, fat-rich cells using cold is made available, which can be used hygienically and particularly effectively in connection with existing systems for generating cold.

### REFERENCE SYMBOL LIST

1 applicator
2 contact surface
3 surface
4 treatment plate
5 channels
6 heat medium
7 plate
8 stack
9 boundary surface
10 boundary surface
11 insulation layer
12 opening
13 groove
14 frame
15 gasket
16 nonwoven fabric
17 temperature sensor
18 ultrasound vibrator
19 stack
20 screw
21 heat medium
22 electrodes
23 gasket
24 beaded gasket
25 gap
26 transition region
27 housing
28 interior space
29 supply line
30 refrigeration device
31 balancer
32 openings
33 lid
34 handle
35 handle
36 attachment point
37 attachment plate
38 installation opening
39 inflow
40 return flow
41 vacuum hose
42 cable
43 connection channel
44 channel
45 device
46 housing block
47 holder
48 connection hose

1. Applicator for use in connection with a device for the treatment of subcutaneous, fat-rich cells using cold, wherein the applicator (1) has a contact surface (2) that is shaped as the surface (3) of a rigid treatment plate (4), preferably from aluminum or from another material having good heat conductivity.

2. Applicator according to claim 1, wherein channels (5) for conducting a heat medium (6) are provided in the rigid treatment plate (4).

3. Applicator according to claim 1, wherein the rigid treatment plate (4) is connected with a further rigid plate (7), to form a stack (8).

4. Applicator according to claim 1, wherein the channels (5) run in a boundary surface (9, 10) of the two firmly connected plates (4, 7).

5. Applicator according to claim 1, wherein an outer insulation layer (11) that covers the stack (8) is provided, wherein a gap (25) is provided between insulation layer (11) and further rigid plate (7), to which gap vacuum is preferably applied.

6. Applicator according to claim 1, wherein the applicator (1) has at least one opening (12) to which a partial vacuum is applied, disposed in or around the contact surface (2).

7. Applicator according to claim 1, wherein the opening (32) to which a partial vacuum is applied is disposed to end in a circumferential groove (13) that frames the contact surface (2).

8. Applicator according to claim 1, wherein the groove (13) is disposed in a rigid frame (14) that encloses the stack (8).

9. Applicator according to claim 1, wherein a circumferential gasket (15) that frames the contact surface (2) is provided, which gasket is preferably configured so as to be easily replaced.

10. Applicator according to claim 1, wherein the gasket (15) is shaped to partially cover the enclosed contact surface (2) in the direction of the surface, and to run out in wedge shape, and wherein at least one channel (44) directed transverse to the course of the gasket (15) is provided in the transition region (26).

11. Applicator according to claim 1, wherein a temperature sensor (17) is provided, which is preferably disposed on the contact surface (2) or affixed within the plate (4).

12. Applicator according to claim 1, wherein an ultrasound vibrator (18) is provided in the treatment plate (4) and/or wherein the contact surface (2) is configured to be bent in concave shape in at least one direction.

13. Applicator according to claim 1, wherein two treatment plates (4) are connected to form a stack (19) with contact surfaces (2) disposed on both sides.

14. Applicator according to claim 1, wherein it has a housing (27), the interior (28) of which is configured to have a partial vacuum applied to it and in which supply lines (29) end in vacuum-tight manner.

15. Device for the treatment of subcutaneous, fat-rich cells using cold, having a heat medium circuit (30) and an applicator (1) through which a heat medium (6) flows, according to claim 1, for treatment of a living body, wherein the device comprises a refrigeration device (30) for making the heat medium (6) available at a specific temperature, and connecting supply lines (29) to the applicator (1), as well as a balancer (31) that compensates the weight of the applicator.
